(19) United States
(12) Patent Application Publication
(10) Pub. No.: US 2023/0184712 A1
(43) Pub. Date: Jun. 15, 2023

(54) SEMICONDUCTOR SENSOR

(71) Applicant: Murata Manufacturing Co., Ltd., Nagaokakyo-shi (JP)

(72) Inventors: Naruto MIYAKAWA, Nagaokakyo-shi (JP); Shota USHIBA, Nagaokakyo-shi (JP); Ayumi SHINAGAWA, Nagaokakyo-shi (JP); Yuka OKA, Nagaokakyo-shi (JP); Masahiko KIMURA, Nagaokakyo-shi (JP)

(21) Appl. No.: 18/109,311

(22) Filed: Feb. 14, 2023

Related U.S. Application Data
(63) Continuation of application No. PCT/JP2021/030004, filed on Aug. 17, 2021.

(30) Foreign Application Priority Data
Aug. 17, 2020 (JP) 2020-137504

(51) Int. Cl. G01N 27/414

(52) U.S. Cl. CPC G01N 27/4146; G01N 27/414; H01L 29/1606

(57) ABSTRACT

A semiconductor sensor includes an insulating substrate, a semiconductor sheet on the insulating substrate and including graphene or carbon nanotubes, a source electrode and a drain electrode, each being provided on the insulating substrate and electrically coupled to the semiconductor sheet, an oxide film extending over a surface of the semiconductor sheet and including silica, alumina, or a composite oxide of silica and alumina, and a receptor at a surface of the oxide film.

### CROSS REFERENCE TO RELATED APPLICATIONS

This application claims the benefit of priority to Japanese Patent Application No. 2020-137504 filed on Aug. 17, 2020 and is a Continuation Application of PCT Application No. PCT/JP2021/030004 filed on Aug. 17, 2021. The entire contents of each application are hereby incorporated herein by reference.

### BACKGROUND OF THE INVENTION

### 1. Field of the Invention

The present invention relates to a semiconductor sensor.

### 2. Description of the Related Art

Field-effect-transistor (FET)-type sensors have recently been attracting attention as sensors using receptors that specifically interact with target molecules, which are target substances to be detected.

As FET-type sensors, for example, a sensor using carbon nanotubes for a channel formed between a source electrode and a drain electrode (U.S. Patent Application Publication No. 2018/0038815) and a sensor using graphene for a channel (Applied Surface Science 480(2019) p. 709-716) have been reported.

### SUMMARY OF THE INVENTION

Each of the sensors described in U.S. Patent Application Publication No. 2018/0038815 and Applied Surface Science 480(2019) p. 709-716 has an insulating substrate, a carbon-based semiconductor disposed on the insulating substrate and composed of carbon nanotubes or graphene, a source electrode and a drain electrode electrically coupled to the carbon-based semiconductor, a linker molecule adsorbed by a non-covalent bond called a “Π stack” on a surface of the carbon-based semiconductor, and a receptor coupled to the linker molecule.

However, each of the sensors described in U.S. Patent Application Publication No. 2018/0038815 and Applied Surface Science 480(2019) p. 709-716 has the following problems.

The surface of the carbon-based semiconductor is hydrophobic. Thus, for example, the problem of denaturation of the molecule constituting the receptor may occur. U.S. Patent Application Publication No. 2018/0038815 and Applied Surface Science 480(2019) p. 709-716 disclose that 1-pyrenebutanoic acid succinimidyl ester (PBASE) is present as a linker molecule on the hydrophobic surface of a carbon-based semiconductor. However, a monolayer of PBASE is not sufficient to reduce the surface hydrophobicity of carbon-based semiconductors.

The carbon-based semiconductor itself has low insulating properties. Thus, when a gate voltage is applied to the sensor, an unnecessary current flows into the carbon-based semiconductor from the gate electrode. This current causes a redox reaction on the surface of the carbon-based semiconductor. This may result in the deposition of an unnecessary material and the electrolysis of the electrolytic solution to reduce the sensitivity of the sensor.

Preferred embodiments of the present invention provide semiconductor sensors in each of which a receptor is not affected by hydrophobicity of a semiconductor surface and electrical insulation of the surface is ensured.

A semiconductor sensor according to a preferred embodiment of the present invention includes an insulating substrate, a semiconductor sheet located on the insulating substrate and including graphene or carbon nanotubes, a source electrode and a drain electrode, each being provided on the insulating substrate and electrically coupled to the semiconductor sheet, an oxide film extending over a surface of the semiconductor sheet and including silica, alumina, or a composite oxide of silica and alumina, and a receptor at a surface of the oxide film.

According to a preferred embodiment of the present invention, it is possible to provide semiconductor sensors in each of which a receptor is not affected by hydrophobicity of a semiconductor surface and electrical insulation of the surface is ensured.

The above and other elements, features, steps, characteristics and advantages of the present invention will become more apparent from the following detailed description of the preferred embodiments with reference to the attached drawings.

### BRIEF DESCRIPTION OF THE DRAWINGS

FIG. 1 is a schematic cross-sectional view illustrating an example of a semiconductor sensor according to a first preferred embodiment of the present invention.

FIG. 2A is a schematic plan view illustrating an example of a step of providing an insulating substrate, and FIG. 2B is a cross-sectional view taken along line IIB-IIB of FIG. 2A.

FIG. 3A is a schematic plan view illustrating an example of a step of forming a source electrode and a drain electrode, and

FIG. 3B is a cross-sectional view taken along line IIIB-IIIB of FIG. 3A.

FIG. 4A is a schematic plan view illustrating an example of a step of forming a semiconductor sheet, and FIG. 4B is a cross-sectional view taken along line IVB-IVB of FIG. 4A.

FIG. 5A is a schematic plan view illustrating an example of a step of forming an oxide film, and FIG. 5B is a cross-sectional view taken along line VB-VB of FIG. 5A.

FIG. 6 is a schematic cross-sectional view illustrating an example of a step of performing silane coupling treatment.

FIG. 7 is a schematic cross-sectional view illustrating an example of a step of disposing a receptor at a surface of an oxide film.

FIG. 8 is a schematic cross-sectional view illustrating another example of the semiconductor sensor according to the first preferred embodiment of the present invention.

FIG. 9 is a schematic cross-sectional view illustrating an example of a semiconductor sensor according to a second preferred embodiment of the present invention.

FIG. 10 is a schematic cross-sectional view illustrating an example of a semiconductor sensor according to a third preferred embodiment of the present invention.

FIG. 11 is a schematic cross-sectional view illustrating another example of the semiconductor sensor according to the third preferred embodiment of the present invention.

FIG. 12 is a schematic cross-sectional view illustrating an example of a semiconductor sensor according to a fourth preferred embodiment of the present invention.

FIG. 13 is a schematic cross-sectional view illustrating an example of a semiconductor sensor according to a fifth preferred embodiment of the present invention.

FIG. 14 is a schematic cross-sectional view illustrating an example of a semiconductor sensor according to a sixth preferred embodiment of the present invention.

FIG. 15 is a schematic plan view illustrating an example of a semiconductor sensor according to the sixth preferred embodiment of the present invention.

FIG. 16 is a schematic cross-sectional view illustrating an example of a semiconductor sensor according to a seventh preferred embodiment of the present invention.

FIG. 17 is a schematic diagram illustrating an example of the structure of a biosensor including a semiconductor sensor according to a preferred embodiment of the present invention.

FIG. 18 is a graph illustrating the relationship between the gate voltage V_{G} and the source-drain current I_{DS}.

### DETAILED DESCRIPTION OF THE PREFERRED EMBODIMENTS

Semiconductor sensors according to preferred embodiments of the present invention will be described below.

The present invention, however, is not limited to the following preferred embodiments, and can be modified as appropriate and applied without departing from the spirit of the present invention. A combination of two or more individual preferred configurations of the present invention described in the following preferred embodiments is also within the scope of the present invention.

Each preferred embodiment described below is an example, and the configurations illustrated in the different preferred embodiments can be replaced or combined in part. In the second and subsequent preferred embodiments, description of matters common to the first preferred embodiment will be omitted, and only different points will be described. In particular, the same operation and effect of the same configuration will not be sequentially described for each preferred embodiment.

### First Preferred Embodiment

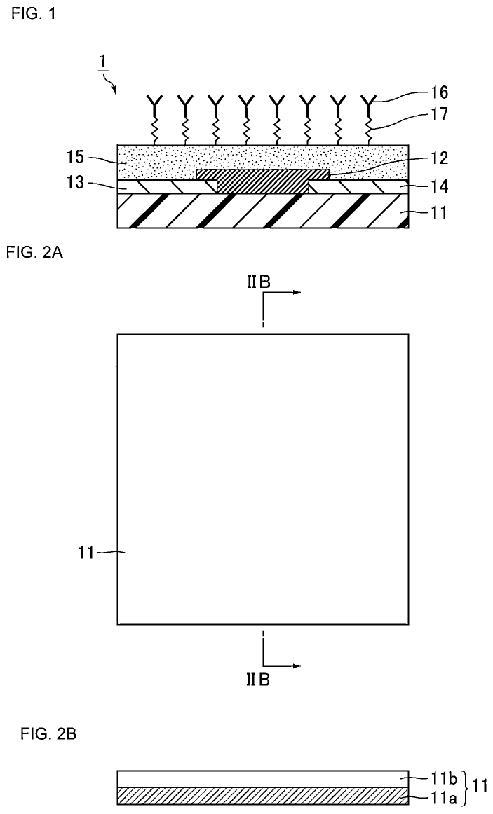

FIG. 1 is a schematic cross-sectional view illustrating an example of a semiconductor sensor according to a first preferred embodiment of the present invention. The thickness of each portion illustrated in FIG. 1 is appropriately changed for clarification and simplification of the drawing. The same applies to other drawings.

A semiconductor sensor 1 illustrated in FIG. 1 includes an insulating substrate 11, a semiconductor sheet 12 located on the insulating substrate 11, a source electrode 13 and a drain electrode 14, each being provided on the insulating substrate 11 and electrically coupled to the semiconductor sheet 12, an oxide film 15 extending over a surface of the semiconductor sheet 12, and a receptor 16 at a surface of the oxide film 15. In the semiconductor sensor 1 illustrated in FIG. 1, the receptor 16 is fixed to the surface of the oxide film 15 with a silane coupling agent 17 interposed therebetween, the silane coupling agent 17 being present on the surface of the oxide film 15.

In the example illustrated in FIG. 1, the source electrode 13 and the drain electrode 14 are positioned on the insulating substrate 11 at a distance from each other. The insulating substrate 11 is exposed between the source electrode 13 and the drain electrode 14. The semiconductor sheet 12 is positioned on the insulating substrate 11 so as to cover an end portion of the source electrode 13, the exposed portion of the insulating substrate 11, and an end portion of the drain electrode 14. The semiconductor sheet 12 between the source electrode 13 and the drain electrode 14 defines the channel of the semiconductor sensor 1.

The insulating substrate 11 is, for example, a thermally oxidized silicon substrate obtained by oxidizing a surface of a silicon (Si) substrate to form a silicon oxide (SiO₂) layer. The material of the insulating substrate 11 is not particularly limited. For example, an inorganic compound, such as silicon oxide, silicon nitride, aluminum oxide, titanium oxide, or calcium fluoride, or an organic compound, such as an acrylic resin, polyimide, or a fluororesin, may be used.

The insulating substrate 11 may be a substrate in which an insulating film is provided on a surface of a conductive substrate. The structures and compositions of the conductive substrate and the insulating film on the conductive substrate are not particularly limited as long as the contact with the semiconductor sheet 12 is insulated by the insulating film.

The shape of the insulating substrate 11 is not particularly limited, and may be a flat plate shape or curved plate shape. The insulating substrate 11 may have flexibility.

The semiconductor sheet 12 includes graphene or carbon nanotubes.

Graphene is a two-dimensional material consisting solely of carbon atoms bonded in a hexagonal mesh (carbon atoms with a honeycomb structure) and having a thickness of one carbon atom, for example. Graphene has a very large specific surface area (surface area per volume) and very high electrical mobility.

However, in this specification, the following carbon-based materials are also broadly defined as graphene.

(1) A carbon-based sheet material in which 2 to 100 layers of graphene are stacked in whole or in part.

(2) The carbon-based sheet material described in (1) above, in which the material is polycrystalline and has grain boundaries.

(3) The carbon-based sheet material described in (2) above, in which the material is partially torn and has an end portion.

(4) The carbon-based sheet material described in any of (1) to (3) above, in which the material is subjected to partial element substitution or has broken honeycomb structure.

(5) Graphene oxide and its reduced graphene oxide.

(6) Ribbon-shaped (strip-shaped) graphene.

(7) Graphene in wrapped form.

(8) A carbon nanotube that is a tubular graphene sheet.

Carbon nanotubes are long tubular carbon compounds. As the carbon nanotubes, single-walled carbon nanotubes (SW-CNTs) each including a single carbon layer having a mesh structure similar to graphene may be used, and multi-walled carbon nanotubes (MW-CNTs) each including many carbon layers stacked may also be used. All of the carbon nanotubes have excellent electrical conductivity.

The source electrode 13 and the drain electrode 14 are, for example, electrodes each having a multilayer structure in which a titanium (Ti) layer and a gold (Au) layer are stacked. With regard to the electrode material, other than titanium and gold, for example, a metal, such as gold, platinum, titanium, or palladium, may be used in the form of a single layer, or a combination of two or more metals may be used in the form of a multilayer structure.

The oxide film 15 includes silica, alumina, or a composite oxide thereof.

The oxide film 15 may include incidental impurities in addition to silica, alumina, or a composite oxide thereof.

The surface of the semiconductor sheet 12 is covered with the oxide film 15 including silica, alumina, or a composite oxide thereof. Thus, the hydrophobicity of the surface of the semiconductor sheet 12 is reduced to render the surface of the semiconductor sensor 1 hydrophilic. As a result, the receptor 16 is not affected by the hydrophobicity of the surface of the semiconductor sheet 12, so that a problem, such as denaturation of the molecule of the receptor 16, can be prevented. A carbon-based semiconductor represented by graphene or a carbon nanotube is a single-element semiconductor and has no polarity in the material. Moreover, the carbon-based semiconductor does not have a chemical bond in any direction other than the directions in which the carbon atoms are bonded together, that is, other than the surface direction. As a result, the carbon-based semiconductor has no polarity in the surface direction and exhibits strong hydrophobicity. In contrast, silica or alumina is a material in which either silicon or aluminum is ionically bonded to oxygen, and has polarity in the material. Moreover, plasma treatment or UV treatment is performed to result in the surface with highly polar hydroxy groups, so that the hydrophilicity can be enhanced. The non-polar semiconductor sheet is covered with such a polar material. This enables the polarization of the surface of the semiconductor sheet to enhance its affinity for water.

Silica and alumina are materials with large band gaps. Thus, the oxide film 15 increases the electrical insulation of the surface of semiconductor sensor 1. This enables suppression of the current flowing from the gate electrode to the semiconductor sheet 12, even when a gate voltage is applied to the semiconductor sensor 1.

The fact that the oxide film 15 includes silica, alumina, or a composite oxide thereof can be verified by subjecting the surface of the semiconductor sensor 1 to elemental analysis using X-ray photoelectron spectroscopy (XPS). Alternatively, it can be verified by subjecting the surface of the semiconductor sensor 1 to elemental analysis using energy dispersive X-ray spectroscopy (EDS).

The oxide film 15 includes silica, alumina, or a composite oxide thereof may be formed of a multilayer body of silica and alumina. The multilayer body of silica and alumina includes one or more silica layers and one or more alumina layers. The number of silica layers and the number of alumina layers may be the same or different.

A protective layer may be provided on the surface of the oxide film 15. The protective layer includes, for example, TiO₂ or ZrO₂. When the protective layer is provided on the surface of the oxide film 15, the corrosion resistance of the oxide film 15 is improved.

In the example shown in FIG. 1, the oxide film 15 extends over the entire insulating substrate 11 and is provided not only on the surface of the semiconductor sheet 12 but also on the source electrode 13 and the drain electrode 14. However, the oxide film 15 may be provided only over the insulating substrate 11 so as to cover at least the surface of the semiconductor sheet 12.

Although the oxide film 15 preferably covers the entire surface of the semiconductor sheet 12, unavoidable defects caused in a production process are allowed. Examples of the unavoidable defects caused in the production process include nonuniformity and chipping of the oxide film 15.

From the viewpoints of ensuring high electrical insulation of the surface of the semiconductor sensor 1 and high mechanical stability of the oxide film 15 (for example, mechanical stability against ultrasonic cleaning), the thickness of the oxide film 15 is preferably about 2 nm or more. The thickness of the oxide film 15 is preferably about 30 nm or less. When the thickness of the oxide film 15 is about 30 nm or less, high sensitivity of the sensor is ensured.

The thickness of the oxide film 15 can be measured by cross-sectional observation with a transmission electron microscope (TEM).

The oxide film 15 preferably includes an amorphous portion. When the oxide film 15 includes the amorphous portion, grain boundaries in the oxide film are reduced. The grain boundaries partially define and function as routes of conductive carriers. Thus, a decrease in the number of grain boundaries results in the partial disappearance of the routes of the conductive carriers. For this reason, when the oxide film 15 includes the amorphous portion, the electrical insulation of the surface of the semiconductor sensor 1 can be enhanced, compared to the case where the oxide film 15 is entirely crystalline. When the oxide film 15 includes the amorphous portion, the entire oxide film 15 does not necessarily have to be amorphous, but may partially include a crystalline region.

The fact that the oxide film 15 includes the amorphous portion can be verified by crystallinity analysis from X-ray diffraction images or electron diffraction images in transmission electron microscope (TEM) measurements.

Examples of the receptor 16 include antibodies, antigens, saccharides, aptamers, or peptides.

The receptor 16 only needs to stay on the surface of the oxide film 15. Only the basal portion of the receptor 16 may be fixed to the oxide film 15, or a portion other than the basal portion may be movable with a certain degree of flexibility.

The presence of the receptor 16 can be verified by the following method. A target substance to be detected corresponding to the receptor 16 is labeled and added to the semiconductor sensor 1. At this time, when the phenomenon that only the labeled target substance to be detected is adsorbed on the semiconductor sensor 1 can be verified, the receptor 16 is considered to be present at the semiconductor sensor 1.

As illustrated in FIG. 1, the receptor 16 is preferably fixed to the surface of the oxide film 15 with the silane coupling agent 17 interposed therebetween, the silane coupling agent 17 being present on the surface of the oxide film 15. In this case, the oxide film 15 and the silane coupling agent 17 are tightly linked together by covalent bonds, and the receptor 16 and the silane coupling agent 17 are tightly linked together by covalent bonds. Thus, the receptor 16 is less likely to be detached, improving the reliability of the sensor.

Examples of the silane coupling agent 17 include amino group-including silane coupling agents, such as 3-aminopropyltriethoxysilane (APTES) and 3-aminopropyltrimethoxysilane (APTMS); thiol group-including silane coupling agents, such as 3-mercaptopropyltriethoxysilane (MPTES); and epoxy group-including silane coupling agents, such as triethoxy(3-glycidyloxypropyl)silane (GPTES).

The presence of the silane coupling agent 17 on the surface of the oxide film 15 can be verified by surface analysis using time-of-flight secondary ion mass spectrometry (TOF-SIMS).

An example of a method for producing the semiconductor sensor according to the first preferred embodiment of the present invention will be described below.

FIG. 2A is a schematic plan view illustrating an example of a step of providing an insulating substrate, and FIG. 2B is a cross-sectional view taken along line IIB-IIB of FIG. 2A.

The insulating substrate 11 is provided as illustrated in FIGS. 2A and 2B. As the insulating substrate 11, for example, a thermally oxidized silicon substrate obtained by oxidizing a surface of a silicon substrate 11a to form a silicon oxide layer 11b is used.

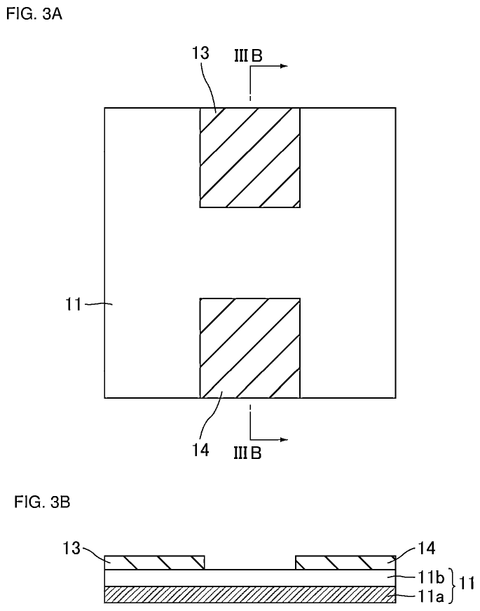

FIG. 3A is a schematic plan view illustrating an example of a step of forming a source electrode and a drain electrode, and FIG. 3B is a cross-sectional view taken along line IIIB-IIIB of FIG. 3A.

For example, a Ti layer and an Au layer are formed on the insulating substrate 11 by a vacuum deposition method, an electron beam (EB) deposition method, a sputtering method, or the like. The layers are then patterned by photolithography and etching to form the source electrode 13 and the drain electrode 14.

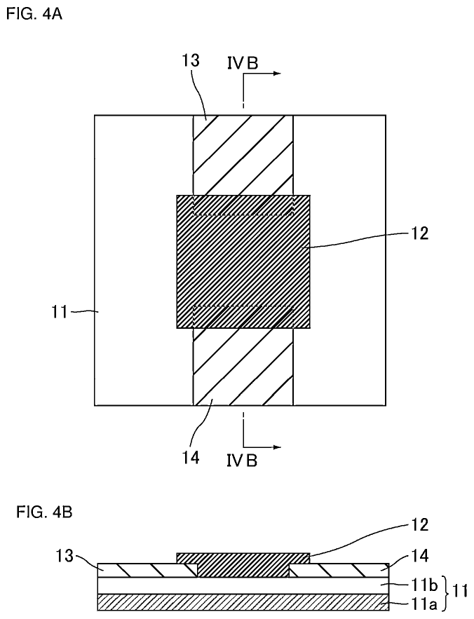

FIG. 4A is a schematic plan view illustrating an example of a step of forming a semiconductor sheet, and FIG. 4B is a cross-sectional view taken along line IVB-IVB of FIG. 4A.

Graphene or carbon nanotubes can be grown on copper foil. For example, graphene or carbon nanotubes grown on the copper foil can be transferred to the insulating substrate 11 and then patterned by photolithography and etching to form the semiconductor sheet 12 on the insulating substrate 11. In the example illustrated in FIGS. 4A and 4B, the semiconductor sheet 12 is formed on the insulating substrate 11 so as to cover an end portion of the source electrode 13 and an end portion of the drain electrode 14.

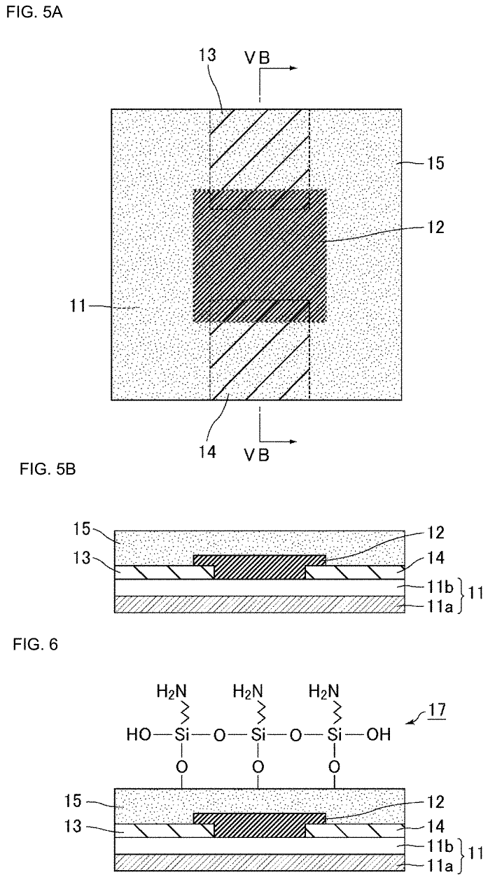

FIG. 5A is a schematic plan view illustrating an example of a step of forming an oxide film, and FIG. 5B is a cross-sectional view taken along line VB-VB of FIG. 5A.

The oxide film 15 including silica, alumina, or a composite oxide thereof is formed by, for example, an atomic layer deposition (ALD) method or an EB deposition method. In the example illustrated in FIGS. 5A and 5B, the oxide film 15 is formed to extend over the entire insulating substrate 11 and provided not only on the surface of the semiconductor sheet 12 but also on the source electrode 13 and the drain electrode 14. The FlexAL (available from Oxford Instruments) can be used for the atomic layer deposition (ALD) method. The PMC-800 (available from Shincron Co., Ltd.) or SEC-10D (available from Showa Shinku Co., Ltd.) can be used for the EB deposition method.

FIG. 6 is a schematic cross-sectional view illustrating an example of a step of performing silane coupling treatment.

As illustrated in FIG. 6, the surface of the oxide film 15 is preferably subjected to the silane coupling treatment. In the example illustrated in FIG. 6, APTES is used as the silane coupling agent 17, and amino groups are present at the surface.

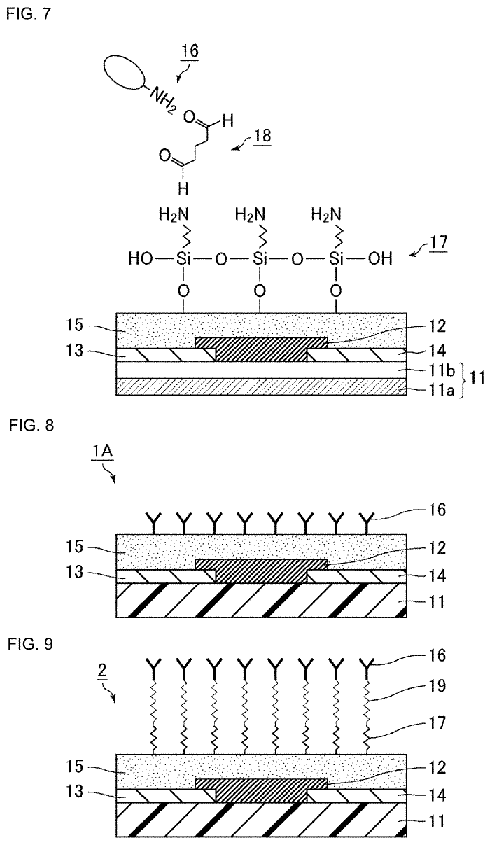

FIG. 7 is a schematic cross-sectional view illustrating an example of a step of disposing a receptor at the surface of an oxide film.

For example, as illustrated in FIG. 7, a fixing agent 18 is fixed to the surface, and then the receptor 16 is provided at the surface of the oxide film 15. In the example illustrated in FIG. 7, glutaraldehyde is used as the fixing agent 18. An amino group of the silane coupling agent 17 and an aldehyde group of the fixing agent 18 are linked by a covalent bond, and the amino group of the receptor 16 and an aldehyde group of the fixing agent 18 are linked by a covalent bond.

Through the above steps, a semiconductor sensor, such as the semiconductor sensor 1 illustrated in FIG. 1, is obtained.

FIG. 8 is a schematic cross-sectional view illustrating another example of the semiconductor sensor according to the first preferred embodiment of the present invention.

As in the semiconductor sensor 1A illustrated in FIG. 8, the receptor 16 may be fixed directly to the surface of the oxide film 15 without using the silane coupling agent 17 (see FIG. 1).

### Second Preferred Embodiment

In a second preferred embodiment of the present invention, a receptor is fixed to a surface of an oxide film with spacer molecules interposed therebetween, the spacer molecules being present at the surface of the oxide film. The spacer molecules allow the receptor at the surface of the oxide film to be spaced apart from the surface of the oxide film and obtain flexibility, thus improving the sensing ability of the receptor. When the spacer molecules are hydrophilic, the hydrophilicity of the surface of the semiconductor sensor can be enhanced.

FIG. 9 is a schematic cross-sectional view illustrating an example of a semiconductor sensor according to the second preferred embodiment of the present invention.

In a semiconductor sensor 2 illustrated in FIG. 9, the silane coupling agent 17 is present on a surface of the oxide film 15, and the spacer molecules 19 are also present. The receptor 16 is fixed to the surface of the oxide film 15 with the spacer molecules 19 interposed therebetween, the spacer molecules 19 being present at the surface of the oxide film 15. The spacer molecules 19 and the silane coupling agent 17 are preferably linked by covalent bonds, and the spacer molecules 19 and the receptor 16 are preferably linked by covalent bonds.

Examples of the spacer molecules 19 include polyethylene glycol (PEG), polyvinylpyrrolidone (PVP), dextran, and ethylene glycol bis(succinimidyl succinate). When their ends are modified with functional groups, the spacer molecules 19 and the silane coupling agent 17 can be linked by covalent bonds, and the spacer molecules 19 and the receptor 16 can be linked by covalent bonds. An example of the spacer molecules 19 that allow an amino group-including silane coupling agent to be linked to an amino group-including receptor by covalent bonds is PEG including a succinimide group at each end. The thickness of the layer including the spacer molecules 19 can be measured by cross-sectional observation with a transmission electron microscope (TEM). The thickness of the layer including the spacer molecules 19 in a preferred embodiment of the present invention is preferably, but not necessarily, about 0.7 nm or more and about 10 nm or less.

### Third Preferred Embodiment

In a third preferred embodiment of the present invention, a blocking agent is present together with a receptor on a surface of an oxide film. The blocking agent enhances the hydrophilicity of the surface of the semiconductor sensor.

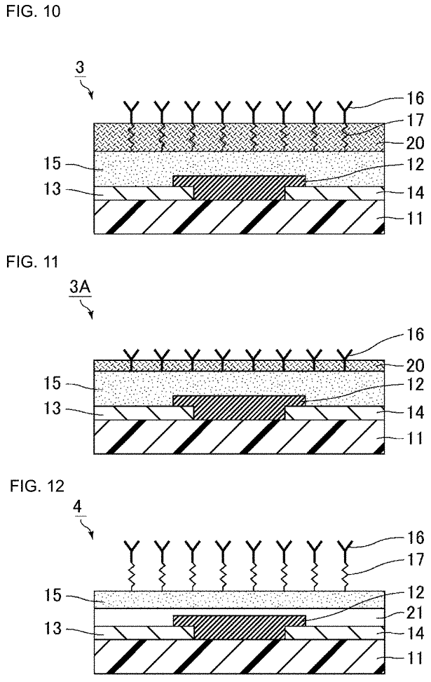

FIG. 10 is a schematic cross-sectional view illustrating an example of a semiconductor sensor according to the third preferred embodiment of the present invention.

In a semiconductor sensor 3 illustrated in FIG. 10, a blocking agent 20 is present on the surface of the oxide film 15, together with the receptor 16. In the semiconductor sensor 3 illustrated in FIG. 10, the receptor 16 is fixed to the surface of the oxide film 15 with the silane coupling agent 17 interposed therebetween, the silane coupling agent 17 being present on the surface of the oxide film 15. The receptor 16 may be fixed to the surface of the oxide film 15 with the spacer molecules 19 interposed therebetween, the spacer molecules 19 being present at the surface of the oxide film 15.

FIG. 11 is a schematic cross-sectional view illustrating another example of the semiconductor sensor according to the third preferred embodiment of the present invention.

As in a semiconductor sensor 3A illustrated in FIG. 11, the receptor 16 may be fixed directly to a surface of the oxide film 15 without using the silane coupling agent 17 (see FIG. 10).

Examples of the blocking agent 20 include proteins (for example, bovine serum albumin (BSA), hemoglobin, and skimmed milk), surfactants (for example, Tween (trade name), Triton (trade name), and sodium dodecyl sulfate (SDS)), and polymers (for example, PEG and PVP).

### Fourth Preferred Embodiment

In a fourth preferred embodiment of the present invention, a seed layer is provided between a semiconductor sheet and an oxide film. When the seed layer is formed, the oxide film is uniformly formed, thus enhancing the sensitivity of the semiconductor sensor.

FIG. 12 is a schematic cross-sectional view illustrating an example of a semiconductor sensor according to the fourth preferred embodiment of the present invention.

In a semiconductor sensor 4 illustrated in FIG. 12, a seed layer 21 is provided between the semiconductor sheet 12 and the oxide film 15. In the example illustrated in FIG. 12, the seed layer 21 is also provided between the semiconductor sheet 12 and the source electrode 13 and between the semiconductor sheet 12 and the drain electrode 14.

The seed layer 21 can be formed, for example, by depositing a light metal, such as aluminum (Al) or magnesium (Mg), a 3d-transition metal, such as titanium (Ti), nickel (Ni), or chromium (Cr), or a rare metal, such as hafnium (Hf), zirconium (Zr), or yttrium (Y), in the form of an elemental metal and then oxidizing the metal.

The thickness of the seed layer 21 is preferably about 2 nm or less. The thickness of the seed layer 21 is preferably about 0.5 nm or more.

In the semiconductor sensor 4 illustrated in FIG. 12, the receptor 16 is fixed to the surface of the oxide film 15 with the silane coupling agent 17 interposed therebetween, the silane coupling agent 17 being present on the surface of the oxide film 15. The receptor 16 may be fixed to the surface of the oxide film 15 with the spacer molecules 19 interposed therebetween, the spacer molecules 19 being present at the surface of the oxide film 15. Alternatively, the receptor 16 may be fixed directly to the surface of the oxide film 15 without using the silane coupling agent 17.

The blocking agent 20 (see FIG. 10) may be present on the surface of the oxide film 15, together with the receptor 16.

### Fifth Preferred Embodiment

In a fifth preferred embodiment of the present invention, an oxide film includes a surface with irregularities. The irregularities on the surface of the oxide film can result in an increase in the surface area of the oxide film, thus enhancing the hydrophilicity of the surface of the semiconductor sensor. Moreover, the density of the receptor molecules arranged on the surface of the oxide film can be increased, thus enhancing the sensitivity of the semiconductor sensor.

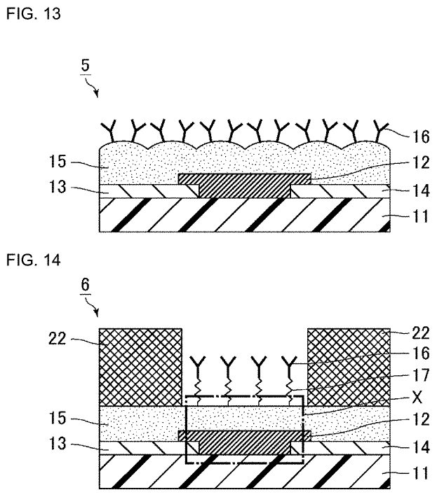

FIG. 13 is a schematic cross-sectional view illustrating an example of a semiconductor sensor according to the fifth preferred embodiment of the present invention.

In a semiconductor sensor 5 illustrated in FIG. 13, the oxide film 15 includes a surface with irregularities.

Examples of a method for forming irregularities on the surface of the oxide film 15 include a method in which the surface is roughened by surface blasting or plasma asking, and a method in which the oxide film is grown in an island configuration. Growth in an island configuration is a phenomenon in which nuclei that are separated from each other grow as starting points. The film is formed non-uniformly by the growth in an island configuration, thus forming irregularities on the surface of the film. An example of a requirement for island growth is that the surface of an underlying film of the film to be grown has poor wettability to the raw material of the film to be grown.

In the semiconductor sensor 5 illustrated in FIG. 13, the receptor 16 is fixed directly to the surface of the oxide film 15 without using the silane coupling agent 17. The receptor 16 may be fixed to the surface of the oxide film 15 with the silane coupling agent 17 interposed therebetween, the silane coupling agent 17 being present on the surface of the oxide film 15. Alternatively, the receptor 16 may be fixed to the surface of the oxide film 15 with the spacer molecules 19 interposed therebetween, the spacer molecules 19 being present at the surface of the oxide film 15.

The blocking agent 20 (see FIG. 10) may be present on the surface of the oxide film 15, together with the receptor 16. The seed layer 21 (see FIG. 12) may be disposed between the semiconductor sheet 12 and the oxide film 15.

### Sixth Preferred Embodiment

In a sixth preferred embodiment of the present invention, an insulating coating layer is provided on a portion of an oxide film other than a sensing portion. When the insulating coating layer is provided, the insulation of the portion other than the sensing portion is enhanced, thus improving the reliability of the semiconductor sensor. Moreover, target molecules are not captured at the portion other than the sensing portion, thus enhancing the sensitivity of the semiconductor sensor.

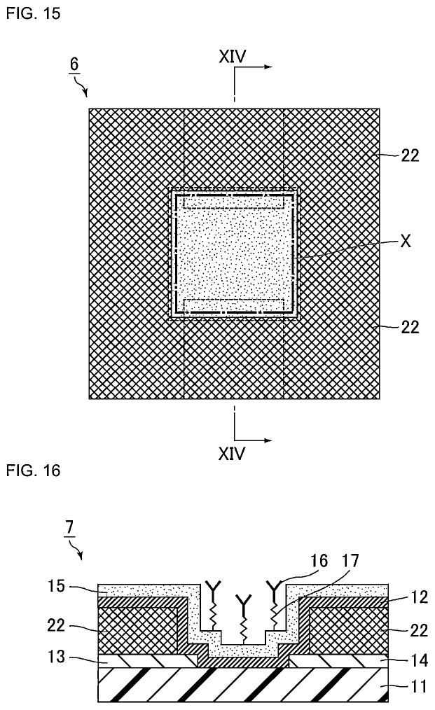

FIG. 14 is a schematic cross-sectional view illustrating an example of a semiconductor sensor according to the sixth preferred embodiment of the present invention. FIG. 15 is a schematic plan view illustrating the example of the semiconductor sensor according to the sixth preferred embodiment of the present invention. FIG. 14 is a cross-sectional view taken along line XIV-XIV of FIG. 15.

In a semiconductor sensor 6 illustrated in FIGS. 14 and 15, the insulating coating layer 22 is provided on a portion of the oxide film 15 other than a sensing portion X. As illustrated in FIG. 15, the insulating coating layer 22 covers the periphery of the oxide film 15 in plan view.

Examples of a material of the insulating coating layer 22 include organic compounds, such as polyimide, epoxy resins, acrylic resins, and fluororesins. The thickness of the insulating coating layer 22 is preferably about 100 nm or more and about 10 μm or less.

In the example illustrated in FIG. 15, although the insulating coating layer 22 is provided on the entire portion of the oxide film 15 other than the sensing portion X, a portion where the insulating coating layer 22 is not provided may be present.

In the semiconductor sensor 6 illustrated in FIG. 14, the receptor 16 is fixed to a surface of the oxide film 15 with the silane coupling agent 17 interposed therebetween, the silane coupling agent 17 being present on the surface of the oxide film 15. The receptor 16 may be fixed to the surface of the oxide film 15 with the spacer molecules 19 interposed therebetween, the spacer molecules 19 being present at the surface of the oxide film 15. Alternatively, the receptor 16 may be fixed directly to the surface of the oxide film 15 without using the silane coupling agent 17.

The blocking agent 20 (see FIG. 10) may be present on the surface of the oxide film 15, together with the receptor 16. The seed layer 21 (see FIG. 12) may be provided between the semiconductor sheet 12 and the oxide film 15. The oxide film 15 may include a surface with irregularities (see FIG. 13).

### Seventh Preferred Embodiment

In a seventh preferred embodiment of the present invention, an insulating coating layer is provided on a source electrode and a drain electrode, and a semiconductor sheet is provided on the source electrode, the drain electrode, and the insulating coating layer.

FIG. 16 is a schematic cross-sectional view illustrating an example of a semiconductor sensor according to the seventh preferred embodiment of the present invention.

In a semiconductor sensor 7 illustrated in FIG. 16, the insulating coating layer 22 is provided on the source electrode 13 and the drain electrode 14, and the semiconductor sheet 12 is provided on the source electrode 13, the drain electrode 14, and the insulating coating layer 22.

A material of the insulating coating layer 22 is the same as in the sixth preferred embodiment.

In the semiconductor sensor 7 illustrated in FIG. 16, the receptor 16 is fixed to a surface of the oxide film 15 with the silane coupling agent 17 interposed therebetween, the silane coupling agent 17 being present on the surface of the oxide film 15. The receptor 16 may be fixed to the surface of the oxide film 15 with the spacer molecules 19 interposed therebetween, the spacer molecules 19 being present at the surface of the oxide film 15. Alternatively, the receptor 16 may be fixed directly to the surface of the oxide film 15 without using the silane coupling agent 17.

The blocking agent 20 (see FIG. 10) may be present on the surface of the oxide film 15, together with the receptor 16. The seed layer 21 (see FIG. 12) may be provided between the semiconductor sheet 12 and the oxide film 15. The oxide film 15 may include a surface with irregularities (see FIG. 13).

### Biosensor

A semiconductor sensor according to a preferred embodiment of the present invention can be used, for example, as a biosensor. In this case, specific examples of a target substance to be detected include cells, microorganisms, viruses, proteins, enzymes, nucleic acids, and low-molecular-weight biological substances.

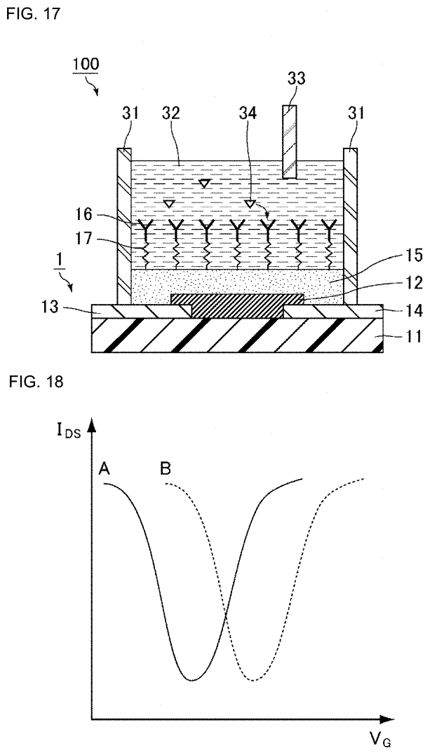

FIG. 17 is a schematic diagram illustrating an example of the structure of a biosensor including a semiconductor sensor according to a preferred embodiment of the present invention.

A biosensor 100 illustrated in FIG. 17 includes the semiconductor sensor 1 illustrated in FIG. 1. The biosensor 100 includes a pool 31 that is mounted on the semiconductor sensor 1 and that includes, for example, silicone rubber, the inside of the pool 31 being filled with an electrolytic solution 32, a gate electrode 33 of the semiconductor sensor 1, the gate electrode 33 being immersed in the electrolytic solution 32, and a bipotentiostat (not illustrated) coupled to the source electrode 13, the drain electrode 14, and the gate electrode 33 of the semiconductor sensor 1. The electrolytic solution 32 includes a target substance 34 to be detected.

The gate electrode 33 is used to apply a potential to the source electrode 13 and the drain electrode 14, and is typically composed of a precious metal. The gate electrode 33 is located at a position other than the positions where the source electrode 13 and the drain electrode 14 are disposed. The gate electrode 33 is usually provided on the insulating substrate 11 or at a place other than the insulating substrate 11. In the semiconductor sensor according to a preferred embodiment of the present invention, however, the gate electrode 33 is preferably above the source electrode 13 or the drain electrode 14.

FIG. 18 is a graph illustrating the relationship between the gate voltage V_{G} and the source-drain current I_{DS}.

In FIG. 18, the source-drain current I_{DS} when the receptor is not bound to the target substance to be detected is indicated by solid line A, and the source-drain current I_{DS} when the receptor is bound to the target substance to be detected is indicated by broken line B. As illustrated in FIG. 18, when the receptor is specifically bound to the target substance to be detected, the conduction characteristics are modulated by the charge of the target molecules, which are the target substance to be detected. By observing the modulation, it is possible to sense the presence or absence or the concentration of the target substance to be detected.

The semiconductor sensors of the present invention is not limited to the above-described preferred embodiments, and various applications and modifications can be made within the scope of the present invention in terms of the configuration of the semiconductor sensors and the production conditions thereof. For example, the silane coupling agent 17 can be replaced with another material that forms a covalent bond on the oxide film 15. Specific examples of such a material include phosphonic acid derivatives.

While preferred embodiments of the present invention have been described above, it is to be understood that variations and modifications will be apparent to those skilled in the art without departing from the scope and spirit of the present invention. The scope of the present invention, therefore, is to be determined solely by the following claims.

What is claimed is:

1. A semiconductor sensor, comprising:
an insulating substrate;
a semiconductor sheet on the insulating substrate and including graphene or carbon nanotubes;
a source electrode and a drain electrode, each being provided on the insulating substrate and electrically coupled to the semiconductor sheet;
an oxide film extending over a surface of the semiconductor sheet and including silica, alumina, or a composite oxide of silica and alumina; and
a receptor at a surface of the oxide film.

2. The semiconductor sensor according to claim 1, wherein at least a portion of the oxide film has a thickness of about 2 nm or more and about 30 nm or less.

3. The semiconductor sensor according to claim 1, wherein the oxide film includes an amorphous portion.

4. The semiconductor sensor according to claim 1, wherein the receptor is fixed to the surface of the oxide film with a silane coupling agent interposed therebetween, the silane coupling agent being present on the surface of the oxide film.

5. The semiconductor sensor according to claim 1, wherein the receptor is fixed to the surface of the oxide film with a spacer molecule interposed therebetween, the spacer molecule being present at the surface of the oxide film.

6. The semiconductor sensor according to claim 1, wherein a blocking agent is present together with the receptor on the surface of the oxide film.

7. The semiconductor sensor according to claim 1, wherein a seed layer is located between the semiconductor sheet and the oxide film.

8. The semiconductor sensor according to claim 1, wherein the oxide film includes a surface with irregularities.

9. The semiconductor sensor according to claim 1, wherein an insulating coating layer is located on a portion of the oxide film other than a sensing portion.

10. The semiconductor sensor according to claim 1, wherein an insulating coating layer is located on the source electrode and the drain electrode; and
the semiconductor sheet is located on the source electrode, the drain electrode, and the insulating coating layer.

11. The semiconductor sensor according to claim 1, wherein the semiconductor sheet covers an end portion of the source electrode, an end portion of the drain electrode, and an exposed portion of the insulating substrate between the source electrode and the drain electrode.

12. The semiconductor sensor according to claim 1, wherein a portion of the semiconductor sheet between the source electrode and the drain electrode defines a channel.

13. The semiconductor sensor according to claim 1, wherein the insulating substrate includes a conductive substrate and an insulating film on the conductive substrate.

14. The semiconductor sensor according to claim 1, wherein each of the source electrode and the drain electrode includes a multilayer structure including at least two layers.

15. The semiconductor sensor according to claim 1, wherein the oxide film includes silica, alumina, or a composite oxide of silica and alumina.

16. The semiconductor sensor according to claim 1, wherein the oxide film includes a multilayer body including silica and alumina.

17. The semiconductor sensor according to claim 1, further comprising a protective layer provided on the oxide film.

18. The semiconductor sensor according to claim 1, wherein the oxide film covers only the surface of the semiconductor sheet.

19. The semiconductor sensor according to claim 1, wherein a thickness of the oxide film is about 2 nm or more and about 30 nm or less.

20. The semiconductor sensor according to claim 1, wherein the receptor includes antibodies, antigens, saccharides, aptamers, or peptides.
